(19) United States
(12) Patent Application Publication
(10) Pub. No.: US 2024/0407851 A1
(43) Pub. Date: Dec. 12, 2024

(54) SURGICAL SYSTEM AND METHOD FOR FORMING LESS THAN ALL BONE CUT SURFACES FOR IMPLANT PLACEMENT

(71) Applicant: Think Surgical, Inc., Fremont, CA (US)

(72) Inventors: Kyle Kuznik, Fremont, CA (US); Micah Forstein, Fremont, CA (US); Joel Zuhars, Fremont, CA (US)

(73) Assignee: Think Surgical, Inc., Fremont, CA (US)

(21) Appl. No.: 18/698,975

(22) PCT Filed: Oct. 10, 2022
(86) PCT No.: PCT/US2022/046146

§ 371 Date: Apr. 5, 2024

Related U.S. Application Data
(60) Provisional application No. 63/253,923, filed on Oct. 8, 2021.

(51) Int. Cl. A61B 34/20; A61B 17/16; A61B 34/10; A61B 34/30

(52) U.S. Cl. CPC A61B 34/20; A61B 17/1675; A61B 34/10; A61B 34/30; A61B 2034/107

(57) ABSTRACT

A method to assist in forming one or more cut surfaces on a bone in preparation for contact with one or more contact surfaces of an implant is provided. Cutting instructions are provided to direct a computer-assisted surgical (CAS) device during formation of a first number of cut surfaces on the bone. The first number of cut surfaces being less than a total number of contact surfaces of the implant. A system for the practice of the method is also provided.

### RELATED APPLICATIONS

This application claims priority benefit of U.S. Provisional Application Ser. No. 63/253,923 filed 8 Oct. 2021; the contents of which are hereby incorporated by reference.

### TECHNICAL FIELD

The present invention generally relates to computer-assisted surgery, and more particularly to a surgical system and method to assist in the preparation of a bone to receive an implant that is unsupported by the surgical system. The surgical system is used to form a number of cut surfaces that is less than the number of implant contact surfaces and a user forms additional cut surfaces without the surgical system.

### BACKGROUND

Throughout a lifetime, bones and joints become damaged and worn through normal use, disease, and traumatic events. Arthritis is a leading cause of joint damage that over time leads to cartilage degradation, pain, stiffness, and bone loss. Arthritis can also cause the muscles articulating the joints to lose strength and become painful.

If the pain associated with the dysfunctional joint is not alleviated by less-invasive therapies, a joint arthroplasty procedure is considered as a treatment. Joint arthroplasty is an orthopedic procedure in which an arthritic or dysfunctional joint surface is replaced with an orthopedic implant.

The accurate placement and alignment of an implant is a large factor in determining the success of joint arthroplasty. In total knee arthroplasty (TKA), the articulating surfaces of the knee joint are replaced with prosthetic components, or implants, typically formed of metal or plastic, to create new articulating joint surfaces. The implants include surfaces intended to contact bone. TKA requires the removal of worn or damaged articular cartilage and bone on the distal femur and proximal tibia, where the removal cuts the bone to provide surfaces (“cut surfaces”) on the remaining bone to contact the contact surfaces of the implant. The position and orientation (POSE) of the cut surfaces determine the final placement and POSE of the implants within the joint. Generally, surgeons plan and create the cut surfaces so the final placement of the implants restores the mechanical axis or kinematics of the patient's leg while preserving the balance of the surrounding knee ligaments. Even small implant alignment errors outside of clinically acceptable ranges correlate with worse outcomes and increased rates of revision surgery.

Current TKA implants are designed to be installed using specific manual instrumentation (e.g., cutting jigs, cutting blocks, alignment fixtures) to form the cut surfaces. Femoral implants typically have five femoral contact surfaces and one or more stabilizing features (e.g., pegs, boxes). The five femoral contact surfaces are intended to contact five cut surfaces on the remaining femur. The stabilizing features of a femoral implant may include pegs or a box to stabilize the femoral implant on the femur. The pegs or box are intended to be inserted into stabilizing cut features (e.g., holes) cut into the bone, typically through a cut surface of the femur and are typically formed perpendicular to a cut surface. Tibial implants typically have one tibial contact surface and a stabilizing feature (e.g., a keel). The one tibial contact surface is intended to contact one cut surface on the remaining tibia. The stabilizing features of a tibial implant may include a keel to stabilize the tibial implant on the tibia. The keel is intended to be inserted into a stabilizing cut feature (e.g., a hole) that is cut into the bone, typically through the cut surface of the tibia, and is typically formed perpendicular to the cut surface. FIGS. 1A-1C illustrate a patient's distal femur 10 and a contour matching femoral implant 12 for a TKA procedure, where five contact surfaces on the implant are intended to contact five cut surfaces on the femur. The anterior cut surface 14 is intended to contact the anterior contact surface 13, the anterior chamfer cut surface 16 is intended to contact the anterior chamfer contact surface 15, the distal cut surface 18 is intended to contact the distal contact surface 17, the posterior chamfer cut surface 20 is intended to contact the posterior chamfer contact surface 19, and the posterior cut surface 22 is intended to contact the posterior contact surface 21. The femoral implant 12 also includes stabilizing features in the form of pegs (23, 24) intended to be inserted into stabilizing cut features (e.g., holes, not shown) formed into the distal cut surface 18 of the femur 10. The manual instrumentation is aligned and affixed to the bone to guide the formation of each cut surface. One of the key struggles associated with manual instrumentation is the proper alignment on the bone. The user has to reference various anatomical landmarks and adjust the cutting jigs based on measurements to ensure the instruments are properly aligned. Aligning a cutting jig to form the femoral distal cut surface and a tibial proximal cut surface are particularly difficult. Once the cutting jig is aligned however, the femoral distal cut surface and tibial proximal cut surface are relatively easy to form by guiding a surgical saw through the cutting jig slots. An additional cutting jig (e.g., a 4-in-1 cut block) is then placed on the femoral distal cut surface to form the remaining cut surfaces on the femur.

Computer-assisted surgery (CAS) can overcome many of the obstacles associated with manual instrumentation. Computer-assisted surgery is an expanding field having applications in total joint arthroplasty (TJA), unicondylar knee arthroplasty, bone fracture repair, maxillofacial reconstruction, and spinal reconstruction. CAS systems are particularly useful for surgical procedures requiring dexterity, precision, and accuracy, and generally include planning software and a computer-assisted surgical device. For example, the TSolution One® Total Knee Application System (“TSolution One Surgical System”, THINK Surgical, Inc., Fremont, CA) aids in the planning and execution of total knee arthroplasty (TKA). The TSolution One Surgical System includes: i) planning software to permit a user to plan the position and orientation of a chosen knee implant model relative to a three-dimensional (3-D) bone model of the patient's femur and tibia; and ii) a surgical robot that precisely mills the bone to form cut surfaces on the bone corresponding to the implant contact surfaces such that the position and orientation of the implant following TKA corresponds to the position and orientation of the implant model as planned by the surgeon.

During the planning phase, the planning software is used to build a 3-D bone model of the patient's bone using pre-operative images (e.g., computed tomography (CT) scans, magnetic resonance imaging (MRI)). The planning software includes an implant library having a plurality of implant models that may be positioned relative to the 3-D bone models to designate the best fit, fill, and/or alignment of the implants on the bones. A user may therefore select an implant model of their choice in the implant library and plan the procedure accordingly.

All of the implants available in the implant library are supported by the CAS system. The CAS system uses the known geometry of each supported implant to accurately cut the bone in the proper shape, position, and orientation to form the cut surfaces on the bone corresponding to the implant contact surfaces to allow the implant to be mounted to the bone in the planned position and orientation.

However, surgeons have widely different preferences on the types of implants they routinely use. If the CAS system does not support an implant then a surgeon cannot use that implant with the CAS system. Likewise, different implant lines may be more beneficial to one patient than another. A patient cannot benefit from the use of the CAS system if the implant needed for the patient is not supported by the CAS system. Thus, there exists a need for a CAS system and method that will allow a CAS system to be used with an unsupported implant.

### SUMMARY OF THE INVENTION

A method to assist in forming one or more cut surfaces on a bone in preparation for contact with one or more contact surfaces of an implant is provided. Cutting instructions are provided to direct a computer-assisted surgical (CAS) device during formation of a first number of cut surfaces on the bone. The first number of cut surfaces being less than a total number of contact surfaces of the implant.

A system is also provided in which components thereof function to provide cutting instructions to direct a CAS device to form a first number of cut surfaces on a bone. The first number of cut surfaces being less than the total number of contact surfaces of an implant.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1A depicts a femur with cut surfaces and a femoral implant with contact surfaces intended to contact the cut surfaces.

FIGS. 1B and 1C depict the femoral implant of FIG. 1A in varying orientations to display the contact surfaces and stabilizing features, where FIG. 1B is a side view thereof and

FIG. 1C is a perspective view thereof.

FIG. 2 illustrates a method of forming cut surfaces on a bone.

FIG. 3 depicts planning software that permits a user to plan positions for one or more cut surfaces to be formed on a bone with a computer-assisted surgical device.

FIG. 4 depicts cutting instructions having a plurality of cut paths that define a cut volume.

FIG. 5 depicts cutting instructions tailored to the shape of a patient's bone.

FIG. 6 depicts a surgical robot forming a femoral distal cut surface on a femur.

FIG. 7 depicts a surgical robot forming a tibia proximal cut surface on a tibia according to a surgical plan.

FIGS. 8A and 8B depict a 4-in-1 cut block to assist in forming cut surfaces on a bone, where FIG. 8A is a top perspective view thereof and FIG. 8B is a bottom perspective view thereof.

FIG. 9 depicts a 4-in-1 cut block aligned on a femoral distal cut surface of a femur.

FIG. 10 depicts a femoral bone including all cut surfaces corresponding to contact surfaces of an implant intended to contact the bone.

FIG. 11 depicts a surgical robot forming a femoral distal cut surface and a femoral posterior cut surface.

FIG. 12 depicts a 4-in-1 cut block aligned on a femoral distal cut surface of a femur.

FIG. 13A depicts a femur having cut surfaces corresponding to contact surfaces of a femoral unicondylar implant intended to contact bone.

FIG. 13B depicts a femoral unicondylar implant.

FIG. 14 depicts a tibia having a cut surface corresponding to a contact surface of a tibial unicondylar implant intended to contact bone.

FIG. 15 depicts planning software that permits a user to plan positions for one or more cut surfaces to be formed on a femur and tibia with a computer-assisted surgical device, where the femur and tibia are subject to a unicondylar knee arthroplasty procedure.

FIG. 16 depicts a surgical robot forming a distal cut surface on a femoral condyle.

FIG. 17 depicts a femoral unicondylar cut block aligned on a distal cut surface formed on a femoral condyle.

FIG. 18 depicts a computer-assisted surgical system that assists in the preparation of a bone to receive an implant.

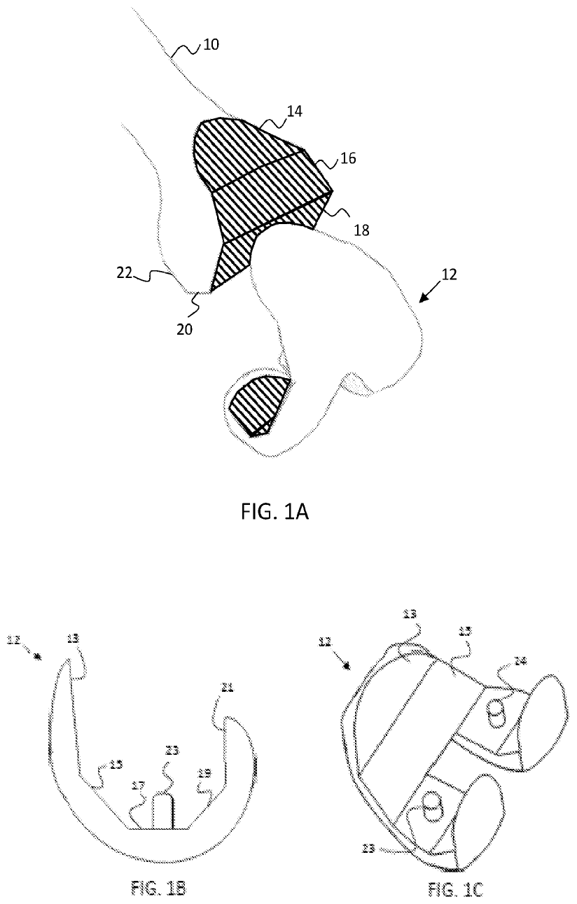

### DETAILED DESCRIPTION

The present invention has utility to allow for the use of unsupported implants with a computer-assisted surgical (CAS) system. The CAS system provides accuracy of planned implant placement on the bone in important degrees-of-freedom and may reduce the overall surgical time.

The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Furthermore, it should also be appreciated that although the systems and methods described herein provide examples with reference to total knee arthroplasty and unicondylar knee arthroplasty, the systems and methods of the present invention may be applied to other computer-assisted surgical procedures involving other joints in the body. By way of example but not limitation, the system and method of the present invention may be applied to the joints of the hip, ankle, shoulder, spine, jaw, elbow, wrist, hands, fingers, feet, toes, etc., as well as revision of initial repair or replacement of any joints or bones. Furthermore, it should be appreciated that the systems and methods may be adapted to form planar cut surfaces and/or non-planar cut surfaces on a bone corresponding to planar and/or non-planar contact surfaces of an implant. References made to contact between cut surfaces on a bone and contact surfaces of an implant when the implant is installed on the bone include contact between the two with and without intermediary substances (e.g., bone cement). Therefore, contact includes contact between cut surfaces on a bone and contact surfaces of an implant without an intermediary substance between the two and contact between cut surfaces on a bone and contact surfaces of an implant with an intermediary substance between the two.

All publications, patent applications, patents and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the terms “computer-assisted surgical device” and “CAS device” refer to devices used in surgical procedures that are at least in part assisted by one or more computers. Examples of CAS devices illustratively include tracked/navigated instruments and surgical robots. Examples of a surgical robot illustratively include robotic hand-held devices, serial-chain robots, bone mounted robots, parallel robots, or master-slave robots, as described in U.S. Pat. Nos. 5,086,401; 6,757,582; 7,206,626; 8,876,830; and 8,961,536; and U.S. Patent Publication No. 2013/0060278; which patents and patent application are incorporated herein by reference. The surgical robot may be active (e.g., automatic/autonomous control), semi-active (e.g. a combination of automatic and manual control), haptic (e.g., tactile, force, and/or auditory feedback), and/or provide power control (e.g., turning a robot or a part thereof on and off). It should be appreciated that the terms “robot” and “robotic” are used interchangeably herein. The terms “computer-assisted surgical system” and “CAS system” refer to systems utilizing a computer or software to assist in planning a position for one or more cut surfaces on a bone or for forming one or more cut surfaces on a bone. An example of a CAS system may include: i) software used by a CAS device (e.g., cutting instructions, pre-operative bone data); ii) software used with a CAS device (e.g., surgical planning software); iii) one or more CAS devices (e.g., a surgical robot); iv) a combination of i), ii), and iii); and iv) any of the aforementioned with additional devices or software (e.g., a tracking system, tracked/navigated instruments, tracking arrays, bone pins, a rongeur, an oscillating saw, a rotary drill, manual cutting guides, manual cutting blocks, manual cutting jigs, etc.). A particular embodiment of a CAS system is described below.

As used herein, the term “digitizer” refers to a device capable of measuring, collecting, recording, and/or designating the location of physical locations (e.g., points, lines, planes, boundaries, etc.) or bone structures in three-dimensional space. By way of example but not limitation, a “digitizer” may be: a “mechanical digitizer” having passive links and joints, such as the high-resolution electro-mechanical sensor arm described in U.S. Pat. No. 6,033,415 (which U.S. patent is hereby incorporated herein by reference); a non-mechanically tracked digitizer probe (e.g., optically tracked, electromagnetically tracked, acoustically tracked, and equivalents thereof) as described for example in U.S. Pat. No. 7,043,961 (which U.S. patent is hereby incorporated herein by reference); an end-effector of a robotic device; or a laser scanner.

As used herein, the term “digitizing” refers to the collecting, measuring, designating, and/or recording of physical locations or bone structures in space with a digitizer.

As used herein, the term “pre-operative bone data” refers to data related to one or more bones, where that data is determined prior to making modifications to the one or more bones. In the case of a revision surgery, pre-operative bone data refers to data related to a bone on which an implant is mounted, where that data is determined prior to the revision surgery. The pre-operative bone data may include: the shapes of the one or more bones; the sizes of the one or more bones; angles and axes associated with the one or more bones (e.g., epicondylar axis of the femoral epicondyles, longitudinal axis of the femur, the mechanical axis of the femur or tibia); angles and axes associated with two bones relative to one another (e.g., the mechanical axis of the knee); and anatomical landmarks associated with the one or more bones (e.g., femoral head center, knee center, ankle center, tibial tuberosity, epicondyles, most distal portion of the femoral condyles, most proximal portion of the femoral condyles). By way of example, the pre-operative bone data may include one or more of the following: an image data set of one or more bones (e.g., an image data set acquired via computed tomography (CT), magnetic resonance imaging (MRI), ultrasound, x-ray, laser scan, etc.); three-dimensional (3-D) bone models, which may include a virtual generic 3-D model of the bone, a physical 3-D model of the bone, a virtual patient-specific 3-D model of the bone generated from an image data set of the bone; and a set of data collected directly on the bone intra-operatively commonly used with imageless CAS devices (e.g., laser scanning the bone, painting the bone with a digitizer).

As used herein, an “end-effector” is a device or tool that interacts with the target object or material (e.g., bone, bone cement). Examples of an end-effector include, but not limited to, a cutter, a burr, end-mill, reamer, drill bit, pin, screw, cutter, saw, laser, and a water-jet.

As used herein, the term “real-time” refers to the processing of input data within fractions of a millisecond to hundreds of milliseconds such that calculated values are available within 2 seconds of computational initiation.

As used herein, the term “cutting instructions” refer to software instructions that direct a CAS device during formation of one or more cut surfaces on a bone. Cutting instructions may further include other instructions, such as instructions for directing the CAS device during formation of bone cuts for stabilizing features of implants (e.g., pegs, boxes, keels). Examples of “cutting instructions” include a cut-file, virtual boundaries, or virtual paths. A “cut-file” may include instructions (e.g., end-effector cut paths, points, orientations, feed rates, or spindle speeds, and any combination thereof as well as other factors) that direct the CAS device during the formation of the cut surfaces on the bone automatically (e.g., a surgical robot executes the instructions to control movement of an end-effector). It should be appreciated that cut-files may be generated with the aid of computer-aided manufacturing (CAM) software. The “cutting instructions” may be virtual boundaries defined relative to the bone position which direct a CAS device to provide feedback (e.g., active, semi-active, haptic, or power control) to a user to assist in the prevention of cutting bone beyond the virtual boundary while the user maneuvers an end-effector of the CAS device during the formation of the cut surfaces. The “cutting instructions” may be virtual paths defined relative to the bone position, which direct a CAS device to provide feedback (active, semi-active, haptic, or power control) to a user to assist in maintaining an end-effector of the CAS device along the virtual path while the user maneuvers the end-effector during the formation of the cut surfaces.

Also referenced herein is a “surgical plan”. A surgical plan is generated, either pre-operatively or intra-operatively, using planning software. The planning software may be used to plan the position for one or more cut surfaces to be formed on the bone based on pre-operative bone data and/or bone data determined intra-operatively. For example, the planning software may be used to generate three-dimensional (3-D) models of the patient's bones from a computed tomography (CT), magnetic resonance imaging (MRI), x-ray, ultrasound image data set, or from a set of points collected on the bone intra-operatively. The planning software may include various software tools to allow a user to plan (i) a position for an implant model on a 3-D bone model for supported implants, which would define the positions of one or more cut surfaces to be formed on the bone, and/or (ii) the positions of one or more cut surfaces to be formed on the bone for unsupported implants, as further described below. As a result, the generated surgical plan may include cutting instructions that will direct the CAS device during formation of the one or more cut surfaces on the bone such that those cut surfaces are made in the desired position and orientation as planned by the user.

As used herein, the term “registration” refers to: the determination of the spatial relationship between two or more objects; the determining of a coordinate transformation between two or more coordinate systems associated with those objects; and/or the mapping of an object onto another object. Examples of objects routinely registered in an operating room (OR) illustratively include: CAS systems/devices; anatomy (e.g., bone); pre-procedure data (e.g., 3-D virtual bone models); a surgical plan (e.g., cut surfaces defined relative to pre-operative bone data, cutting instructions defined relative to pre-operative bone data); and any external landmarks (e.g., a tracking array affixed to a bone, an anatomical landmark, a designated point/feature on a bone, etc.) associated with the bone (if such landmarks exist). Methods of registration known in the art are described in U.S. Pat. Nos. 6,033,415; 8,010,177; 8,036,441; and 8,287,522; and U.S. Patent Application Publication 2016/0338776. In particular embodiments with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a 3-D bone model. The points collected from the surface of a bone with the digitizer may be matched using iterative closest point (ICP) algorithms to generate a transformation matrix. The transformation matrix provides the correspondence between: i) the position of the bone in an operating room (OR); ii) the bone model and the cutting instructions associated with the bone model); and iii) the CAS device.

Also used herein is the term “optical communication” which refers to wireless data transfer via infrared or visible light that are described in U.S. Pat. No. 10,507,063 and assigned to the assignee of the present application.

Also referenced herein are “unsupported implants”. An implant is unsupported by a CAS system if the CAS system is unable to provide cutting instructions to a CAS device for all of the cut surfaces on a bone needed to correspond to all of the contact surfaces of an implant. An implant may be unsupported if any of the following apply: i) the CAS system does not include a model of the implant; ii) the CAS system does not include or is unable to create cutting instructions based on the geometry of the implant; and/or iii) the CAS system lacks knowledge of the geometry of the implant (e.g., data pertaining to the geometry of the implant is unknown by the CAS system).

To overcome the lack of implant geometrical information, certain key cut surfaces may be planned relative to pre-operative bone data (e.g., 3-D bone models, anatomical landmarks, axes, and angles) to define several important alignment degrees-of-freedom for the unsupported implant on the bone. For example, in TKA, the key cut surfaces may include one or more of the femoral distal cut surface, the femoral posterior cut surface, and the tibial proximal cut surface. The CAS device is able to safely form these key cut surfaces on the bone using cutting instructions created based on general shapes, virtual boundaries, or virtual paths as further described below. The general shapes may be patient-sized but not specific to the geometry of an implant. Thus, the planning software and the CAS device advantageously provide precision and accuracy in planning and forming the key cut surfaces to establish several important alignment degrees-of-freedom for the unsupported implant on the bone, while the remaining cut surfaces may be formed using manual instrumentation. This allows for an unsupported implant to be used with a CAS system.

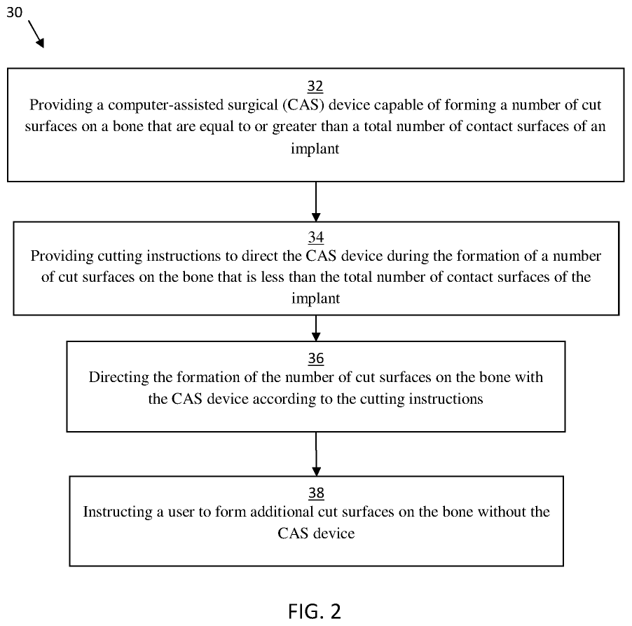

With reference now to the figures, FIG. 2 illustrates a particular embodiment of a method 30 to assist in forming one or more cut surfaces on a bone in preparation for contact with one or more contact surfaces of an implant, where a CAS device is provided that may be capable of being used in the formation of a number of cut surfaces that is equal to or greater than the a total number of contact surfaces of the implant [Block 32] but receives cutting instructions for use in the formation of a number of cut surfaces that is less than a total number of contact surfaces of the implant [Block 34]. The CAS device is then used during the formation of the number of cut surfaces that is less than the total number of contact surfaces of the implant [Block 36] and the user receives instructions for forming the remaining cut surfaces without the CAS device (e.g., with manual instruments) [Block 38]. Specific embodiments of the method and the corresponding systems and devices are described below.

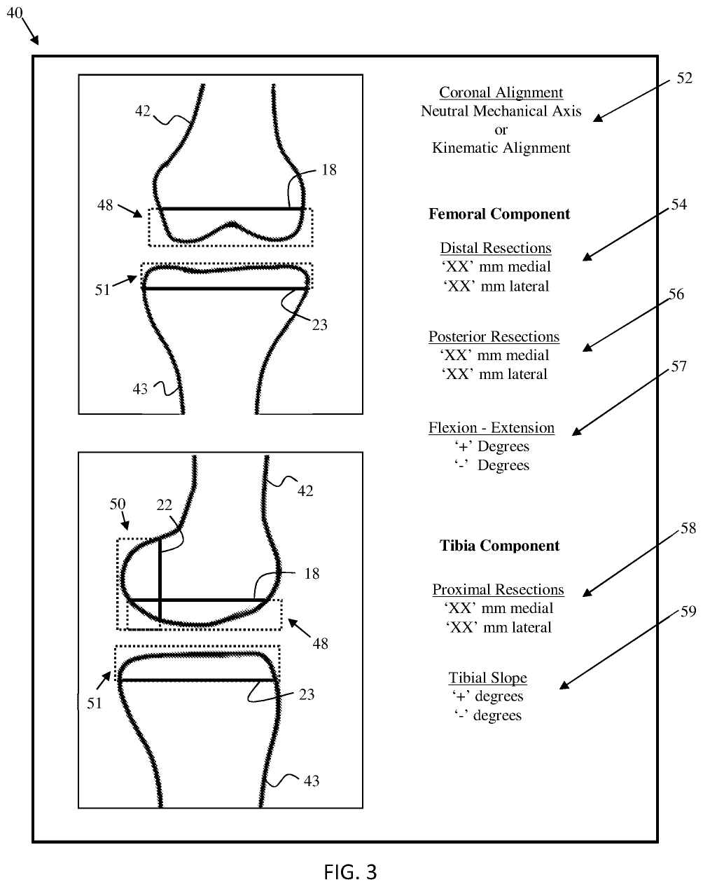

With reference to FIG. 3, planning software is depicted on a monitor 40 for planning a TKA procedure. The planning software may display on a graphical user interface (GUI) one or more views of a 3-D bone model. As examples, a femoral bone model 42 and a tibial bone model 43 are shown in FIG. 3 in both a coronal (top window) and sagittal view (bottom window). The 3-D bone models may be built from pre-operative images (e.g., computed tomography (CT), magnetic resonance imaging (MRI), ultrasound, x-ray, laser scan, or a combination thereof) using image segmentation and computer graphic techniques (e.g., marching cubes) known in the art. The planning software further includes various software tools (52, 54, 56, 57, 58, 59) to assist the user in planning a position for one or more cut surfaces (18, 22, 23) to be formed on the bone relative to the femoral bone model 42 and the tibia bone model 43. For planning a TKA procedure, the user may plan the position for the femoral distal cut surface 18 and the tibia proximal cut surface 23 relative to their respective bone models (42, 43) and the user may also plan the position for the femoral posterior cut surface 22 relative to the femoral bone model 42. The various software tools (52, 54, 56, 57, 58, 59) may allow the user to select their desired coronal alignment goal 52 (e.g., neutral mechanical axis alignment, kinematic alignment), the amount of femoral distal resection 54 on the condyles, the amount of femoral posterior resection 56 on the condyles, the degree of flexion-extension 57 for a cut surface on the femur, the amount of tibial proximal resection 58, and the degree of tibial slope 59 for a cut surface on the tibia. The planning software may further include other software tools that allow the user to select, drag, and rotate one or more virtual objects (e.g., a virtual plane, a virtual path, a virtual cut volume (described below), a generic implant model, a general shape) to plan the position for the one or more cut surfaces (18, 22, 23) relative to the 3-D bone models (42, 43).

In a particular embodiment, the planning software may automatically determine one or more degrees-of-freedom for a cut surface relative to the bone models (42, 43) when the user selects a desired coronal alignment goal 52. The user and/or the planning software may identify various anatomical landmarks (e.g., femoral head center, center of the knee, ankle center, trans-epicondylar axis, longitudinal axis of the tibia, etc.) to assist in this determination. For example, if a user selects a neutral mechanical axis alignment goal, then the varus-valgus angle of the femoral distal cut surface 18 may be determined by first defining the mechanical axis of the femur and then defining the varus-valgus angle as being perpendicular to the mechanical axis of the femur. Likewise, the varus-valgus angle of the tibia proximal cut surface 23 may be determined by first defining the mechanical axis of the tibia and then defining the varus-valgus angle as being perpendicular to the mechanical axis of the tibia. Methods for identifying anatomical landmarks, axes, planes and to determine one or more cut surfaces based on clinical alignment goals is described in U.S Pat. Pub. No. 20190090952 assigned to the assignee of the present application.

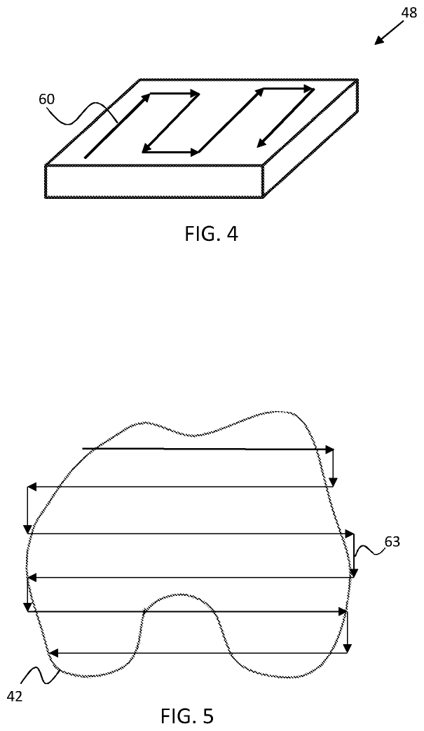

The planning software may further display a representation of the cutting instructions (e.g., cut paths, virtual planes, virtual paths) to assist the user in planning the position for the one or more cut surfaces (18, 22, 23) relative to the 3-D bone models (42, 43). The cutting instructions may be displayed as a cut volume (48, 50, 51) on the GUI of the planning software. A cut volume (48, 50, 51) is a volume that an end-effector of a CAS device traverses (or can traverse) during the formation of one or more cut surfaces (18, 22, 23). For example, as shown in FIG. 4 and further described below, a cut volume 48 may be defined by a plurality of cut paths 60, where a CAS device is instructed to automatically control the end-effector to traverse and cut along the cut paths 60 to form a cut surface 18 (FIG. 3). The cut volume (48, 50, 51) may be defined based on: i) the peripheral cut paths in a cut-file; or ii) a set of virtual boundaries that form a volume in which an end-effector can be manipulated. The user may plan a position for one or more cut surfaces by positioning a cut volume (18, 22, 23) relative to the 3-D bone models (42, 43). For example, with reference to FIG. 3, the user may plan a position for the femoral distal cut surface by positioning a cut volume 61 such that the top plane of the cut volume 61 intersects with the 3-D femoral bone model 42 at the desired femoral distal cut surface 18. FIG. 3 depicts a first cut volume defining the position for the femoral distal cut surface 18, a second cut volume 50 defining the position for the femoral posterior cut surface 22, and a third cut volume 51 defining a position for the tibial proximal cut surface 23.

In a particular embodiment, the planning software automatically positions cutting instructions (48, 50) (e.g., cut paths, virtual boundaries, virtual paths) relative to the 3-D bone models (42, 43) when the user plans the position for a cut surface using other techniques. For example, the user may select a desired coronal alignment goal, distal resection, and flexion-extension angle using the software tools (52, 54, 57) to plan a position for the femoral distal cut surface, where the planning software then automatically positions cutting instructions relative to the 3-D bone model corresponding to those selections.

With reference to FIG. 4, a specific embodiment of a cut volume 48 is shown including a plurality of cut paths 60 which may be defined in a cut-file. With conventional CAS systems, the cut paths are defined based on the geometry of a supported implant. Since the geometry of an unsupported implant is unknown, the cutting instructions may be generalized to permit an unsupported implant to be used with a CAS system. For example, a cut-file may be created having cut paths that define a cut volume 61 in a general shape (e.g., a rectangular prism (as shown in FIG. 4), a polyhedron, an ovoid, a cylinder, a disc, a generic implant shape or portion thereof, etc.). For example, the cut paths 60 may be along a single plane, a single line, or three-dimensional. If the cut paths 60 are along a single plane or line, then the width of the cut volume 61 may be defined by the width or diameter of the end-effector. The cutting instructions (and therefore the cut volumes) may be modifiable to conform to the shape of the patient's bone where the cutting will occur. For example, FIG. 5 depicts a plurality of cut paths 63 that generally match the shape of the patient's distal femur at the location of the femoral distal cut surface. The planning software may include measurement tools for measuring the size of the patient's 3-D bone model where a cut surface is positioned and the cutting instructions may be modified (manually or by the planning software) based on these measurements.

The planning software may further include a library of sets of cutting instructions, where each set of cutting instructions in the library define a cut volume of different shapes and sizes. For example, the library of sets of cutting instructions may include a set of cutting instructions ‘A’ having a cut volume in the shape of a rectangular prism with size ‘X’, set of cutting instructions ‘B’ have a cut volume in the shape of a rectangular prism with size ‘Y’, and set of cutting instructions ‘C’ have a cut volume in the shape of a distal femur with size ‘Z’, etc. Each set of cutting instructions may be in a cut-file that the user or the software selects that is best suited for a particular patient. The cut-files in the library may differ by shape and size to best match the shape and size of the 3-D bone model where the cutting will occur. By selecting the appropriately sized set of instructions ensures the cutting will not occur into the soft tissues or other non-targeted areas surrounding the bone.

In a specific embodiment, the cutting instructions are in the form of virtual boundaries or virtual paths. The virtual boundaries may be defined as a line, plane, or volume. The user may position the virtual boundaries relative to the 3-D bone models using the aforementioned techniques to plan a position for one or more cut surfaces on the bone. For example, referring back to FIG. 3, a user may position a virtual plane relative to the femoral bone model 42 to define the position for the femoral distal cut plane 18. In the OR, the CAS system provides feedback (e.g., force, tactile, audible) to prevent the end-effector from cutting any bone proximal to the distal cut plane 18. The planning software may include a library of different virtual boundary shapes for the user to choose from to best the match the shape and size of the 3-D bone model where the cutting will occur. In another embodiment, the cutting instructions are in the form of one or more virtual paths. The user may position a virtual path relative to the 3-D bone models using the aforementioned techniques to plan a position for one or more cut surfaces on the bone. For example, referring back to FIG. 3, a user may position a virtual plane relative to the femoral bone model 42 to define the position for the femoral distal cut surface 18. In the OR, the CAS system provides feedback (e.g., force, tactile, audible) to maintain the end-effector along the virtual plane to form the femoral distal cut surface 18.

Overall, the planning software provides all the tools necessary to accommodate different user's preferences, approaches, and techniques for planning a position for one or more cut surfaces on a bone to provide planning for the position of an unsupported implant. The planning software allows each user to plan a position for one or more key cut surfaces relative to the bone models as they see fit. While pre-operative bone data and/or bone data gathered intraoperatively is primarily used to plan the position for the one or more cut surfaces, the user may also employ generally known, public, or previous knowledge of the geometry of portions of the implant to assist in planning. For example, it is well known that most femoral implants have a standard distal thicknesses ranging from 8 millimeters (mm) to 10 mm. A user may therefore plan the femoral distal cut surface by: i) using a standard distal thicknesses of a femoral implant to establish the amount of femoral distal resection; ii) determining the mechanical axis of the femur to assist in establishing the varus-valgus angle; and iii) using the distal anterior femoral cortex plane to establish the flexion-extension angle. The tibial proximal cut surface may be planned by: i) using a standard thickness of a tibial implant to establish the amount of tibial proximal resection; ii) determining the mechanical axis of the tibia to assist in establishing the varus-valgus angle; and iii) determining the native posterior slope using the proximal tibial medullary canal and the proximal tibial anterior cortex to establish the posterior slope. It should be appreciated that any anatomical landmarks, anatomical planes, anatomical axes, and anatomical angles may be used to assist the user in planning the position for one or more cut surfaces. The planning software may further include a plurality of generic implant models of varying shape and size. The user may select a generic implant model having a geometry that closely matches with a final implant the user intends to mount on the patient's bone to assist in planning the position for one or more cut surfaces.

After the user has planned the position for the one or more cut surfaces relative to the pre-operative bone data, the surgical plan is transferred/uploaded to the CAS device. The surgical plan includes cutting instructions based on the position of the one or more cut surfaces relative to the 3-D bone model(s), where the CAS device will follow the cutting instructions during formation of the one or more cut surfaces on the bone. For unsupported implants, the surgical plan will only include cutting instructions for a number of cut surfaces, which is less than a total number of contact surfaces of the implant.

The following provides three examples of a system and method for executing an arthroplasty procedure with an unsupported implant. In the first example, the CAS system is used for TKA to plan and form: i) the femoral distal cut surface; and ii) the tibial proximal cut surface. Additional cut surfaces on the femur (e.g., anterior cut surface, posterior cut surface, anterior chamfer cut surface, and posterior chamfer cut surface) will be formed without the CAS device, for example, they may be formed using manual instrumentation. In the second example, the CAS system is used for TKA to plan and form: i) the femoral distal cut surface; ii) the femoral posterior cut surface; and iii) the tibial proximal cut surface. Additional cut surfaces on the femur (e.g., anterior cut surface, anterior chamfer cut surface, and posterior chamfer cut surface) will be formed without the CAS device, for example, they may be formed using manual instrumentation. The third example describes a system and method for executing a unicondylar knee arthroplasty with an unsupported implant.

### Example 1: Femoral Distal Cut Surface and Tibial Proximal Cut Surface for TKA

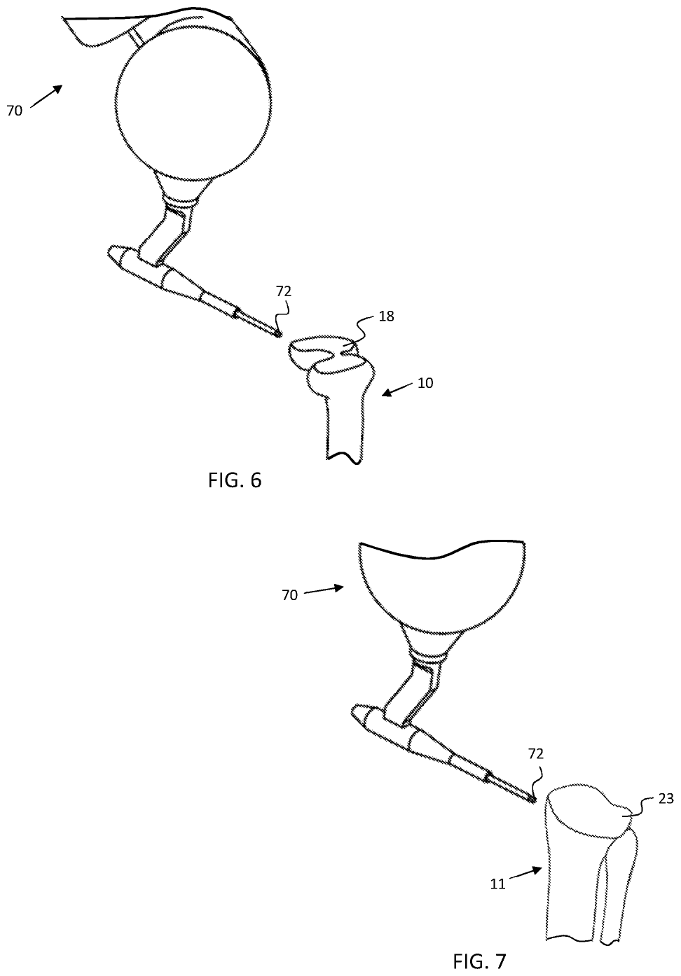

In this example, the surgical plan may include cutting instructions that direct the CAS device during formation of only the femoral distal cut surface 18 and the tibia proximal cut surface 23. In the operating room (OR) the femur and tibia bone(s) are exposed in a traditional manner. The bones are then either: i) fixed relative to the CAS device using fixation equipment (e.g., fixation rods, clamps, pins); or ii) a tracking reference device (e.g., a tracking array) is affixed to the bone to permit a tracking system to track the bone position during the procedure. After which, the surgical plan is registered to the operative bone(s). Registration maps the surgical plan (and therefore the cutting instructions) to the position of the bones to provide the CAS device with the location to form the cut surfaces. The CAS device is then directed by the cutting instructions during the formation of the femoral distal cut surface 18 and the tibia proximal cut surface 23. FIG. 6 depicts a particular embodiment where the femoral distal cut surface 18 is formed by a surgical robot 70. The surgical robot 70 may execute cutting instructions to automatically control an end-effector 72 to form the femoral distal cut surface 18. The same surgical robot 70 may execute cutting instructions to form the tibia proximal cut surface 23 on the tibia 11 as shown in FIG. 7. After the femoral distal cut surface 18 is formed, or both the femoral distal cut surface 18 and the tibia proximal cut surface 23 is formed, the user follows instructions to form additional cut surfaces on the femur without the CAS device (e.g., by using manual instrumentation). By creating the femoral distal cut surface 18 with the CAS device, the following implant alignment degrees-of-freedom on the femur are controlled: i) varus/valgus; ii) proximal/distal; and iii) flexion/extension. By creating the tibia proximal cut surface 23 with the CAS device, the following implant alignment degrees-of-freedom on the tibia are controlled: i) varus/valgus; ii) proximal/distal; and iii) flexion/extension. This is without any other cut surfaces formed on either bone. By controlling these six degrees-of-freedom, the coronal accuracy of the implant placement on the bones is comparable to the coronal accuracy that can be produced by a conventional CAS system and method. Thus, using the CAS device during the formation of these two cut surfaces is highly advantageous from an accuracy, efficiency, and usability perspective over formation of these two cut surfaces with manual instrumentation.

The user will be instructed to form the remaining cut surfaces on the femur without the CAS device. For example, the user may use manual instrumentation to form the remaining cuts. The user may be instructed to form the remaining cut surfaces on the femur in a variety of ways as understood by one of skill in the art. For example, the instructions may be in text on a display monitor, a graphic on a display monitor, a graphical user interface (GUI), an instruction manual, a user's manual, a technical data sheet, release notes, instructions-for-use (IFU's), and equivalents thereof.

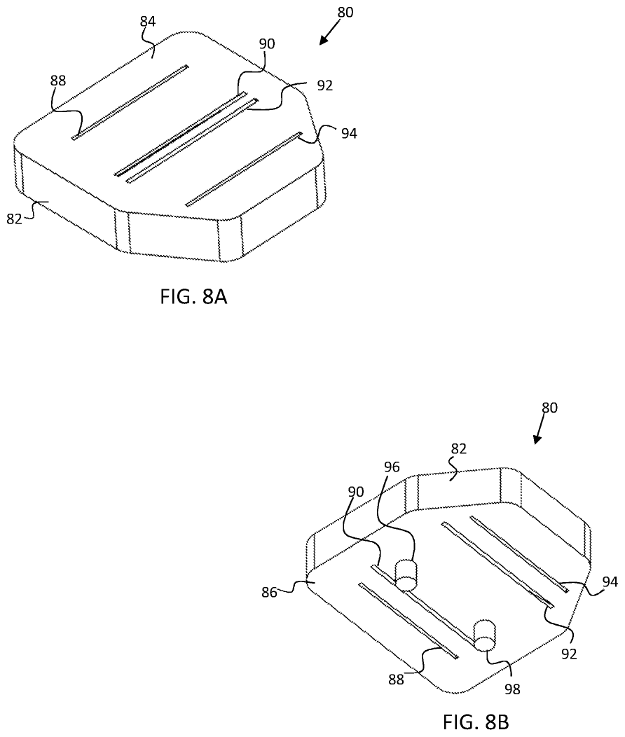

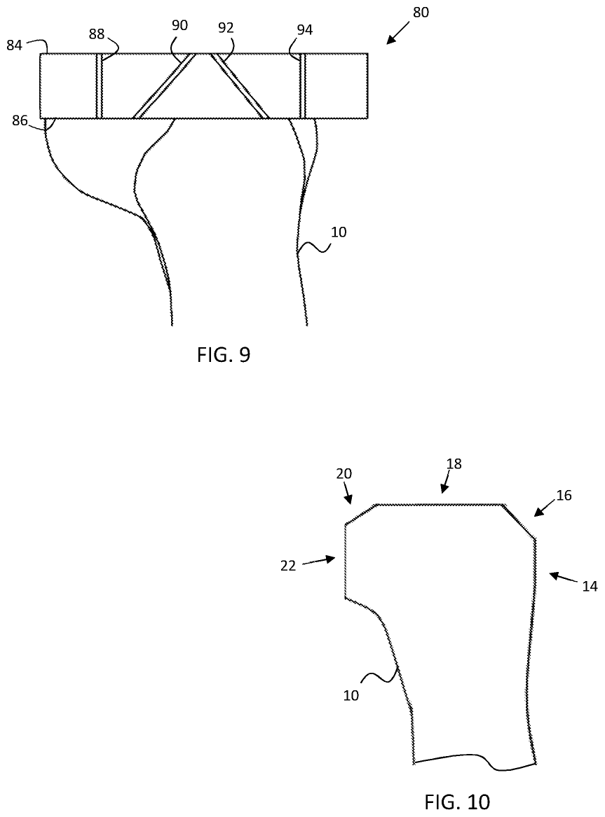

One example of a manual instrument is a 4-in-1 cut block 80 as shown in FIGS. 8A, 8B, and 9, where FIG. 8A depicts a top perspective view of the 4-in-1 cut block 80, FIG. 8B depicts a bottom perspective view of the 4-in-1 cut block 80, and FIG. 9 depicts a side view of the 4-in-1 cut block 80 aligned on the femoral distal cut surface 18. The 4-in-1 cut block 80 includes a body 82 having a top surface 84, and a bottom surface 86 that contacts and lies against the distal cut surface 18 on the femur. The 4-in-1 cut block 80 further includes a plurality of guide slots extending through the body 82 from the top surface 84 to the bottom surface 86. The 4-in-1 cut block 80 in some inventive embodiments includes a posterior guide slot 88, a posterior chamfer guide slot 90, an anterior chamfer guide slot 92, and an anterior guide slot 94. The 4-in-1 cut block 80 may further include a pair of pegs (96 and 98) projecting from the bottom surface 86 where the pair of pegs (96 and 98) fit into corresponding peg holes cut through the distal cut surface 18. The position of the pegs (96 and 98) on the 4-in-1 cut block 80 are at a known geometry relative to the guide slots such that when the 4-in-1 cut block 80 is assembled into the peg holes made on the distal cut surface 18, the guide slots are aligned to aid in the creation of the remaining cut surfaces. Alternative to the pegs (96, 98), the 4-in-1 block may include one or more holes or slots that align with one or more pins or screws to secure the 4-in-1 cut block on the bone. The posterior guide slot 88 and the anterior guide slot 94 are configured to guide a surgical saw to create the posterior cut surface 22 (as shown in FIG. 12) and the anterior cut surface 14 (as shown in FIG. 12), respectively. The posterior chamfer guide slot 90 and the anterior chamfer guide slot 92 are angled and positioned to guide a surgical saw to create the posterior chamfer cut surface 20 (as shown in FIG. 12) and the anterior chamfer cut surface 16 (as shown in FIG. 12), respectively.

The 4-in-1 cut block 80 may be aligned on the distal cut surface 18 (FIG. 9) using conventional techniques. For example, an alignment jig or sizing guide may be used to form the peg holes or insert pins into the distal cut surface to position the 4-in-1 cut block 80 in the proper orientation. However, this alignment is relatively easy compared to the manual alignment of conventional cutting jigs to create the femoral distal cut surface 18. In a specific embodiment, the CAS device may assist in marking the position for the 4-in-1 block on the femoral distal cut surface 18. The user may have prior knowledge of the general shape and geometry of the 4-in-1 block and may enter one or more of these measurements in the planning software such that the CAS device can mark the position of the 4-in-1 block in a desired position/orientation. FIG. 9 depicts a 4-in-1 block 80 aligned on the femoral distal cut surface 18. A user may then guide a surgical saw through the guide slots in the 4-in-1 block 80 to form the remaining cut surfaces (e.g., anterior cut surface 14, anterior chamfer cut surface 16, posterior chamfer cut surface 20, and posterior cut surface 22). FIG. 10 depicts a femur having all the cut surfaces corresponding to the contact surfaces of an implant intended to contact bone.

It should be appreciated that other manual instrumentation may be used to form the remaining cut surfaces and/or assist in the formation of the remaining cut surfaces. Examples of other manual instrumentation include: i) guides having a single guide slot; ii) cutting instruments (e.g., surgical saw, broach); iii) tracked cutting instruments; iv) pins, screws, rods, or clamps; v) a bone mounted guide with a rotatable guide slot; vi) an angle wing to assist with anterior cut surface alignment; and/or vii) other alignment jigs, sizing guides, cut guides, or cut blocks that permit a user to form to the remaining cut surfaces on a bone.

### Example 2: Femoral Distal Cut Surface, Femoral Posterior Cut Surface, and Tibial Proximal Cut Surface for TKA

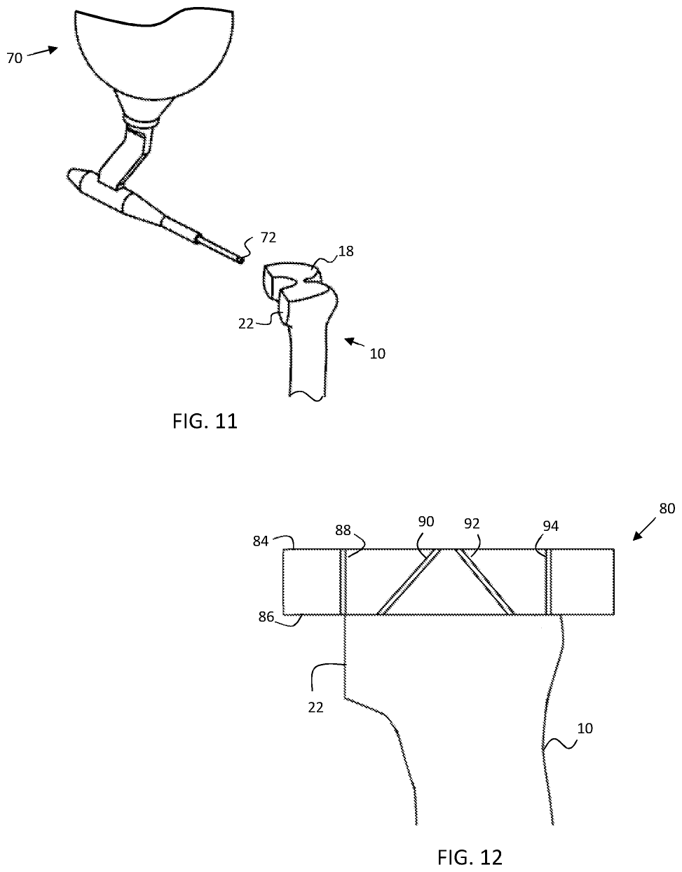

In this example, the surgical plan includes cutting instructions to direct the CAS device during formation of only the femoral distal cut surface 18, the femoral posterior cut surface 22, and the tibia proximal cut surface 23. In the OR the femur and tibia bone(s) are exposed in a traditional manner. The operative bones are then either: i) fixed relative to the CAS device using fixation equipment (e.g., fixation rods, clamps, pins); or ii) a tracking reference device (e.g., a tracking array) is affixed to the bone to permit a tracking system to track the bone position during the procedure. After which, the surgical plan is registered to the operative bone(s). Registration maps the surgical plan (and therefore the cutting instructions) to the position of the bones to provide the CAS device with the location to form the cut surfaces. The CAS device is then directed by the cutting instructions during the formation of the femoral distal cut surface 18, the femoral posterior cut surface 22, and the tibia proximal cut surface 23. FIG. 11 depicts a particular embodiment where the femoral distal cut surface 18 and femoral posterior cut surface 22 is formed by a surgical robot 70. The surgical robot 70 may execute cutting instructions to automatically control an end-effector 72 to form femoral distal cut surface 18 and the femoral posterior cut surface 22 on the femur 10. The same surgical robot 70 may execute cutting instructions to form the tibia proximal cut surface 23 on the tibia 11 as shown in FIG. 7. After the femoral cut surfaces (18, 22) are formed, or both the femoral cut surfaces (18, 22) and the tibia proximal cut surface 23 are formed, the user follows instructions to form additional cut surfaces on the femur without the CAS device (e.g., by using manual instrumentation). By forming the femoral distal cut surface 18 and the femoral posterior cut surface 22 with the CAS device, the following implant alignment degrees-of-freedom on the femur are controlled: i) varus/valgus; ii) proximal/distal; iii) flexion/extension; iv) internal/external rotation; and v) anterior/posterior. By forming the tibia proximal cut surface 23 with the CAS device, the following implant alignment degrees-of-freedom on the tibia are controlled: i) varus/valgus; ii) proximal/distal; and iii) flexion/extension. This is without any other cut surfaces formed on either bone. By controlling these eight degrees-of-freedom, the overall accuracy of the implant placement on the bone is comparable to the overall implant placement accuracy that can be produced by a conventional CAS system and method. Thus, using the CAS device during the formation of these three cut surfaces is highly advantageous from an accuracy, efficiency, and usability perspective over formation of these three cut surfaces with manual instrumentation.

To form the remaining cut surfaces on the femur, the user may use manual instrumentation. In a particular embodiment, a 4-in-1 cut block 80 (as shown in FIGS. 8A, 8B and 12) may be aligned on the distal cut surface 18 using conventional techniques or with the aid of the CAS device as previously described. In a specific embodiment, the femoral posterior cut surface 22 may be used as a guide to align the 4-in-1 cut block 80 on the femoral distal cut surface 18 by aligning the posterior guide slot 88 of the 4-in-1 cut block 80 with the previously formed femoral posterior cut surface 22. FIG. 12 depicts a 4-in-1 cut block 80 aligned on the femoral distal cut surface 18, where the posterior guide slot 88 is aligned with the femoral posterior cut surface 22 previously formed with the CAS device. A user may then guide a surgical saw through the remaining guide slots in the 4-in-1 block 80 to form the remaining cut surfaces (e.g., anterior cut surface 14, anterior chamfer cut surface 16, and posterior chamfer cut surface 20). FIG. 10 depicts the prepared femur after all the cut surfaces have been formed.

### Example 3: Unicondylar Knee Arthroplasty (UKA)

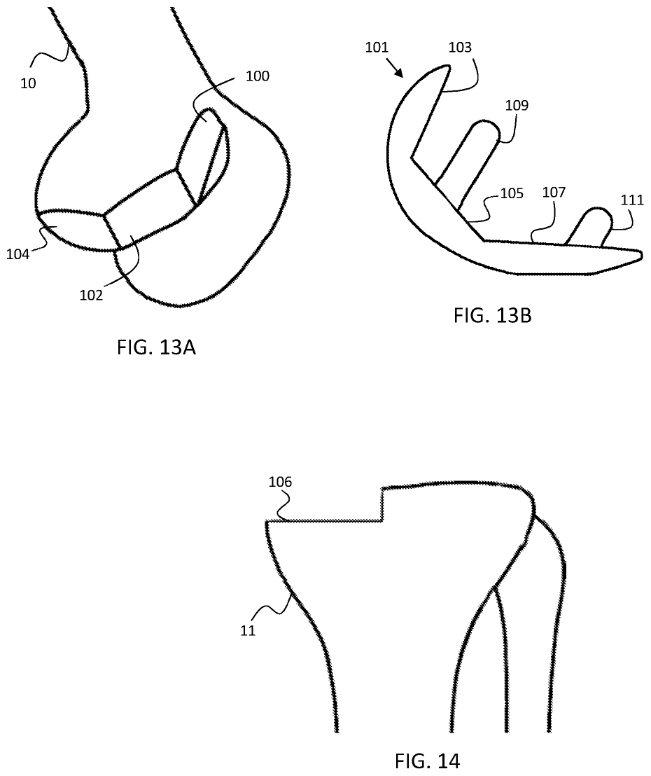

FIGS. 13A and 13B depict a femur 10 and a femoral unicondylar implant 101, where the femur 10 has cut surfaces (100, 102, 104) corresponding to contact surfaces of the femoral unicondylar implant 101 intended to contact bone. This includes the anterior cut surface 100 on a femoral condyle (it could be the medial or lateral side depending on the worn side of the knee) intended to contact the anterior contact surface 103, the distal cut surface 102 on a femoral condyle intended to contact the distal contact surface 105, and the posterior cut surface 104 on a femoral condyle intended to contact the posterior contact surface 107. A posterior chamfer cut surface an anterior chamfer cut surface on the femoral condyle may also be needed depending on the number of contact surfaces of the femoral unicondylar implant. The femoral unicondylar implant 101 further includes pegs (109, 111) to be inserted into holes (not shown) cut into the distal cut surface 102 and posterior cut surface 104. FIG. 14 depicts a tibia having a proximal cut surface 106 on a tibial condyle (it could be the medial or lateral depending on the worn side of the knee) corresponding to a contact surface of a tibial unicondylar implant.

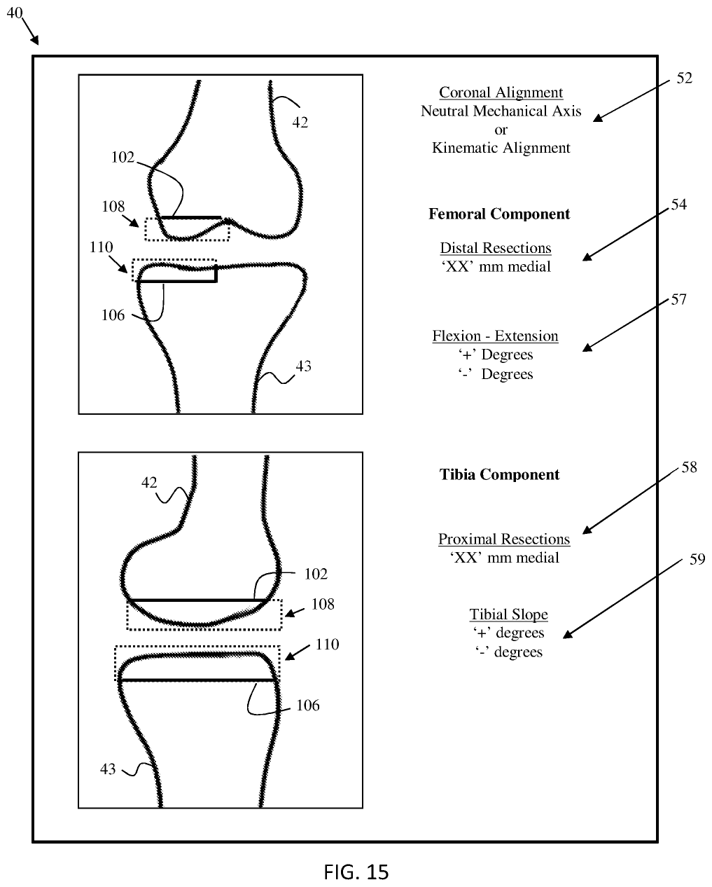

The user may plan a UKA procedure with an unsupported implant using the same techniques as described above. With reference to FIG. 15, planning software is depicted on a monitor 40 displaying a femoral bone model 42 and a tibial bone model 43. The user may plan a position for an unsupported unicondylar implant by planning a position for the distal cut surface on the femoral condyle (in this case the medial condyle) and a proximal cut surface on the tibial condyle using the methods described above. For example, a cut volume (108, 110) may be displayed on the GUI where the user can drag and rotate a cut volume (108, 110) relative to the 3-D bone models (42, 43) to plan the position for the cut surfaces (102, 106) to be formed by the CAS device.

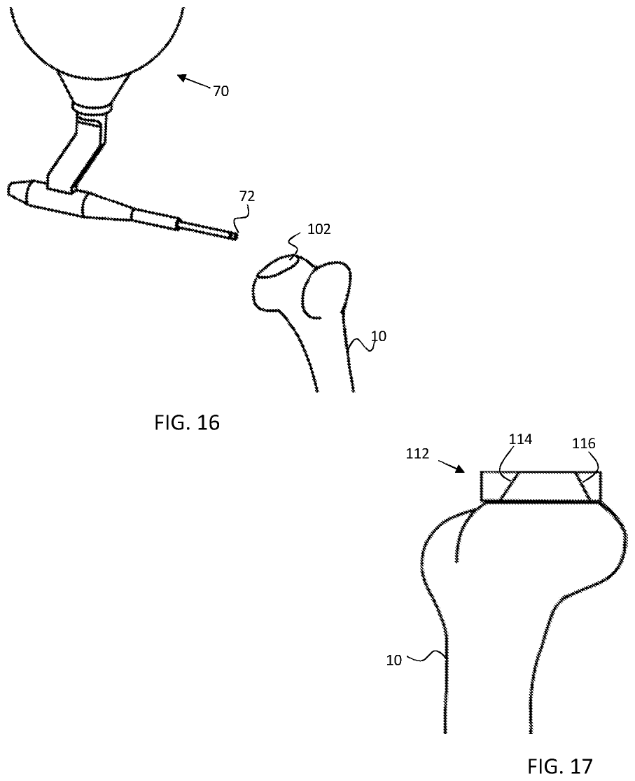

The surgical plan includes cutting instructions that direct the CAS device during formation of the distal cut surface 102 (FIG. 13) on the femoral condyle and the proximal cut surface 106 on the tibial condyle. In the OR the femur and tibia are exposed in a traditional manner. The operative bones are then either: i) fixed relative to the CAS device using fixation equipment (e.g., fixation rods, clamps, pins); or ii) a tracking reference device (e.g., a tracking array) is affixed to the bone to permit a tracking system to track the bone during the procedure. After which, the surgical plan is registered to the position of the operative bone(s). Registration maps the surgical plan (and therefore the cutting instructions) to the position of the bones to provide the CAS device with the location to form the cut surfaces. The CAS device is then directed by the cutting instructions during the formation of the distal cut surface 102 on the femoral condyle according to the surgical plan. FIG. 16 depicts a particular embodiment where the distal cut surface 102 on the femoral condyle is formed by a surgical robot 70. The surgical robot 70 may execute cutting instructions to automatically control an end-effector 72 to form the distal cut surface on the femoral condyle. The same surgical robot 70 may execute cutting instructions to form the proximal cut surface 106 on the tibial condyle (not shown). After the distal cut surface 102 is formed on the femoral condyle, or both the distal cut surface 102 on the femoral condyle and the proximal cut surface 106 and the tibial condyle are formed, the user is instructed to form additional cut surfaces on the femur without the CAS device (e.g., by using manual instrumentation).

To form the remaining cut surfaces on the femur, the user may use manual instrumentation. In a particular embodiment, with reference to FIG. 17, a unicondylar cut block 112 may be aligned on the distal cut surface 102 on the femoral condyle using conventional techniques or with the aid of the CAS device as previously described. The unicondylar femoral cut block 112 may include a plurality of guide slots (114, 116) similar to the 4-in-1 cut block 80 described with reference to FIGS. 8A and 8B. A user may then guide a surgical saw through the guide slots in the unicondylar femoral cut block 112 to form the remaining cut surfaces on the femoral condyle (e.g., anterior cut surface 100 and posterior chamfer cut surface 104). The unicondylar femoral cut block may include additional guide slots for forming a posterior chamfer cut surface and/or an anterior chamfer cut surface if needed.

### Intra-Operative Adjustments

In a specific embodiment, the CAS system allows the user to adjust the position of the one or more cut surfaces in the OR. Since the implant is unsupported by the CAS system, adjustments may be particularly helpful to account for the final geometry of the implant that will be mounted on the patient's bone. A GUI, in the OR, may display an option or drop-down menu that allows the user to adjust the position of a cut surface. For example, a drop-down menu may allow the user to incrementally change the amount of femoral distal resection, the amount of femoral posterior resection, and/or the amount of tibial resection. The adjustments may also allow for a change in the relative angle between one cut surface relative to another. For example, a femoral implant may have a posterior contact surface that is non-perpendicular to the distal contact surface. An option may allow the user to adjust the angle of the posterior cut surface relative to the distal cut surface to account for this non-perpendicularity.

### Computer-Assisted Surgical (CAS) System

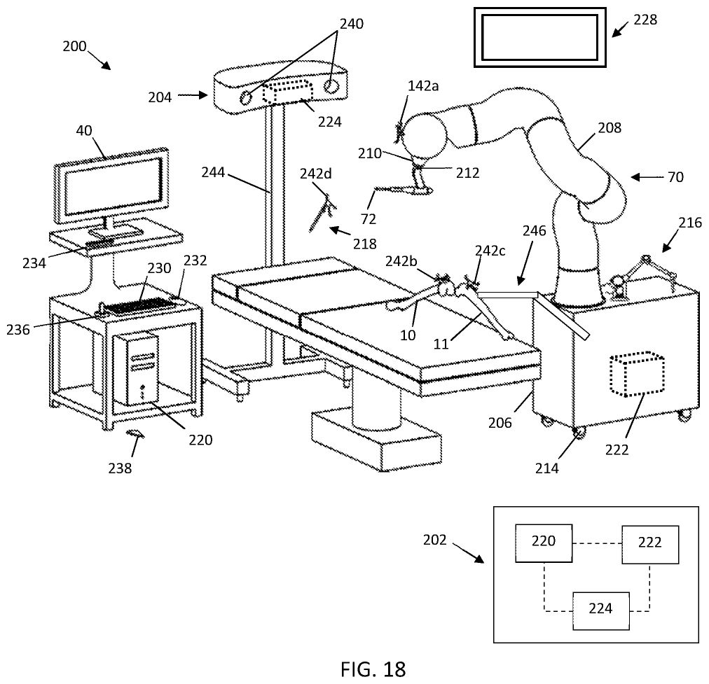

FIG. 18 depicts a particular embodiment of a CAS system in the form of a robotic surgical system 200. The robotic surgical system 200 is shown in the context of an operating room (OR) to prepare a femoral bone 10 and tibial bone 11 for total knee arthroplasty or unicondylar knee arthroplasty. The surgical system 200 includes a surgical robot 70, a computing system 202, and an optional tracking system 204. The surgical robot 70 may include a movable base 206, a robot arm 208 connected to the base 206, an end-effector 72 located at a distal end 210 of the robot arm 208, and a force sensor 212 positioned proximal to the end-effector 72 for sensing forces experienced on the end-effector 72. The base 206 includes a set of wheels 214 to maneuver the base 206, which may be fixed into position using a braking mechanism such as a hydraulic brake. The base 206 may further include an actuator to adjust the height of the robot arm 208. The robot arm 208 includes various joints, links, and sensors (e.g., encoders) to accurately manipulate the end-effector 72 in various degrees of freedom. The joints are illustratively prismatic, revolute, spherical, or a combination thereof. The end-effector 72 may be a motor-driven end-mill, cutter, drill-bit, or other bone removal device. In some embodiments, the surgical system 200 further includes a mechanical digitizer arm 216 attached to the base 206 to assist in digitizing the bones and/or the registration process. In other inventive embodiments, the system includes a tracked hand-held digitizer device 218 with a probe tip to be tracked by the tracking system 204, where the hand-held digitizer device 218 assists in digitizing the bones and/or the registration process.

The computing system 202 may generally include a planning computer 220; a device computer 222; a tracking computer 224 (if present); and peripheral devices. The planning computer 220, device computer 222, and tracking computer 224 may be separate entities, one-in-the-same, or combinations thereof depending on the surgical system. Further, in some embodiments, a combination of the planning computer 220, the device computer 222, and/or tracking computer 224 are connected via a wired or wireless communication. The peripheral devices allow a user to interface with the surgical system components (intraoperatively and/or preoperatively) and may include: one or more user-interfaces, such as a display or monitor (40, 128) to display a graphical user interface (GUI); and user-input mechanisms, such as a keyboard 230, mouse 232, pendent 234, joystick 236, foot pedal 238, or the monitor (40, 228) in some inventive embodiments has touchscreen capabilities.

The planning computer 220 contains hardware (e.g., processors, controllers, and/or memory), software, data and/or utilities that are in some inventive embodiments dedicated to the planning of a surgical procedure, either pre-operatively or intra-operatively. This may include reading pre-operative bone data, displaying pre-operative bone data, manipulating pre-operative bone data (e.g., image segmentation), constructing three-dimensional (3D) virtual bone models, storing supported computer-aided design (CAD) implant models, storing generic unsupported CAD implant models, providing various software tools, functions, and/or widgets to aid a user in planning the surgical procedure, and generating a surgical plan. The generated surgical plan may include: pre-operative bone data (e.g., 3-D virtual bone models); patient identifier data; registration data including the position of a set of points defined relative to the pre-operative bone data for registration; a planned position of one or more cut surfaces relative to the pre-operative bone data; and/or a cutting instructions to be used by the surgical robot 70 to form one or more cut surfaces on the bone as planned. In particular embodiments, the cutting instructions are in a cut-file for execution by a surgical robot to automatically form the cut surfaces on a bone, which is advantageous from an accuracy and usability perspective. The surgical plan generated from the planning computer 220 may be transferred/uploaded to the device computer 222 and/or tracking computer 224 through a wired or wireless connection in the operating room (OR); or transferred via a non-transient data storage medium (e.g., a compact disc (CD), a portable universal serial bus (USB) drive, a dongle with optical communication capabilities) if the planning computer 120 is located outside the OR.

The device computer 222 in some inventive embodiments is housed in the moveable base 206 and contains hardware, software, data and/or utilities that are preferably dedicated to the operation of the surgical robot 70. This may include surgical device control, robotic manipulator control, the processing of kinematic and inverse kinematic data, the execution of registration algorithms, the execution of calibration routines, the execution of cutting instructions, coordinate transformation processing, providing workflow instructions to a user, and utilizing position and orientation (POSE) data from the tracking system 204 (if present).

The tracking system 204 (if present) may be an optical tracking system that includes two or more optical receivers 240 (e.g., cameras) to detect the position of fiducial markers (e.g., retroreflective spheres, active light emitting diodes (LEDs)) uniquely arranged on rigid bodies. The fiducial markers arranged on a rigid body are collectively referred to as a tracking array (242a, 242b, 242c, 242d), where each tracking array has a unique arrangement of fiducial markers, or a unique transmitting wavelength/frequency if the markers are active LEDs. An example of an optical tracking system is described in U.S. Pat. No. 6,061,644. The tracking system 204 may be built into a surgical light, located on a boom, a stand 244, or built into the walls or ceilings of the OR. The tracking system computer 224 may include tracking hardware, software, data, and/or utilities to determine the POSE of objects (e.g., bones B, surgical robot 108, end-effector 72) in a local or global coordinate frame. The POSE of the objects is referred to herein as POSE data, where this POSE data may be communicated to the device computer 222 through a wired or wireless connection. Alternatively, the device computer 222 may determine the POSE data using the position of the fiducial markers detected from the optical receivers 240 directly.

The POSE data is determined using the position data detected from the optical receivers 140 and operations/processes such as image processing, image filtering, triangulation algorithms, geometric relationship processing, registration algorithms, calibration algorithms, and coordinate transformation processing.

The POSE data is used by the computing system 202 during the procedure to update the POSE and/or coordinate transforms of the bone B, the surgical plan, and the surgical robot 70 as the robot arm 208 and/or bone(s) (F, T) move during the procedure, such that the surgical robot 70 can accurately execute the surgical plan.

In specific embodiments, the surgical system 200 does not include a tracking system 104, but instead employs a mechanical digitizer arm 216, and a bone fixation system 246 with bone fixation hardware (e.g., rods, pins, clamps) to fix the bone to the surgical robot 70. A bone motion monitoring system may monitor bone movement when the bones are fixed to the surgical robot 70 as described in U.S. Pat. No. 5,086,401.

The computing system 202 may include one or more processors, non-transient memory, and software for performing embodiments of the methods described herein. In a particular embodiment, the computing system 202 includes software executable instructions that when executed by the processor causes the processor to perform one or more of the following functions: i) provide planning software and a graphical user interface that permits a user to plan the position for one or more cut surfaces relative to pre-operative bone data; ii) provide planning software and a graphical user interface that permit a user to plan a position for cutting instructions relative to pre-operative bone data, the cutting instructions to be used by a CAS device during the formation of one or more cut surfaces on a bone that are less than a total number of contact surfaces of an implant; iii) automatically positioning cutting instructions relative to pre-operative bone data based a planned position of one or more cut surfaces relative to pre-operative bone data; iv) receive a surgical plan having a planned position for cutting instructions relative to pre-operative bone data; v) register a surgical plan to a bone relative to a CAS device; vi) direct a CAS device to form one or more cut surfaces on a bone according to cutting instructions; vii) track the bone and the CAS device during a procedure; and viii) instruct a user to form additional planes on a bone without a CAS device.

CAS Device Capable of Forming Less than all Bone Cut Surfaces for Implant Placement

Embodiments of the present invention may utilize a CAS device capable of forming only a number of cut surfaces on the bone that is less than a total number of contact surfaces of the implant. For example, a CAS device may be designed by structure and/or function to form only the femoral distal cut surface 18 (FIG. 6) on the femur 10. Cutting instructions may direct the CAS device during the formation of only the number of cut surfaces that the CAS device is capable of forming, which is less than the total number of contact surfaces of the implant. The user may form the remaining cut surfaces without the CAS device by using manual instrumentation.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method to assist in forming one or more cut surfaces on a bone in preparation for contact with one or more contact surfaces of an implant, the method comprising:
providing cutting instructions to direct a computer-assisted surgical (CAS) device during formation of a first number of cut surfaces on the bone, wherein the first number of cut surfaces is less than a total number of contact surfaces of the implant.

2. The method of claim 1, further comprising directing the formation of the first number of cut surfaces on the bone with the CAS device according to the cutting instructions.

3. The method of claim 2, further comprising instructing a user to form additional cut surfaces on the bone without the CAS device, or to utilize manual instrumentation to form additional cut surfaces on the bone.

4. (canceled)

5. The method of claim 3, wherein the first number of cut surfaces formed using the CAS device plus the number of additional cut surfaces correspond to the total number of contact surfaces of the implant.

6. The method of claim 1, further comprising using planning software to plan a position for the first number of cut surfaces relative to pre-operative bone data.

7. The method of claim 1, wherein the cutting instructions are part of a total knee arthroplasty (TKA) procedure.

8. (canceled)

9. The method of claim 1, wherein the cutting instructions are part of a unicondylar knee arthroplasty (UKA) procedure.

10. (canceled)

11. (canceled)

12. The method of claim 1, wherein the implant is unsupported by the CAS device.

13. The method of claim 1, wherein the cutting instructions comprise at least one of a cut-file, one or more virtual boundaries, or one or more virtual paths, one or more cut paths situated along a single plane, along a single line, or in three-dimensions, or define a cut volume, wherein the cut volume has a general shape.

14. (canceled)

15. (canceled)

16. The method of claim 1, wherein the CAS device is an active surgical robot, a semi-active surgical robot, or a haptic surgical robot.

17. The method of claim 1, wherein the cutting instructions direct the CAS device during the formation of a second number of cut surfaces on a second bone, wherein the second number of cut surfaces is equal to or less than a total number of contact surfaces of a second implant.

18. (canceled)

19. The method of claim 2 wherein the CAS device is capable of forming a number of cut surfaces on the bone that is equal to or greater than the total number of contact surfaces of the implant.

20. A system comprising:
cutting instructions to direct a CAS device to form a first number of cut surfaces on a bone, wherein the first number of cut surfaces is less than the total number of contact surfaces of an implant.

21. The system of claim 20, further comprising:
a CAS device directed by the cutting instructions during the formation of the first number of cut surfaces on the bone.

22. The system of claim 20, further comprising a mechanism to instruct a user to form additional cut surfaces on the bone without the CAS device, or to complete additional cuts surfaces on the bone with manual instrumentation.

23. (canceled)

24. The system of claim 22, wherein the mechanism comprises at least one of: text on a display monitor; a graphic on a display monitor; a graphical user interface (GUI); an instruction manual; a user's manual; a technical data sheet; release notes; instructions-for-use (IFUs); and equivalents thereof.

25. (canceled)

26. The system of claim 20, further comprising planning software to plan a position for the first number of cut surfaces relative to pre-operative bone data.

27. (canceled)

28. (canceled)

29. (canceled)

30. (canceled)

31. (canceled)

32. (canceled)

33. The system of claim 20, wherein the cutting instructions comprises at least one of a cut-file, one or more virtual boundaries, or one or more virtual paths, one or more cut paths situated along a single plane, along a single line, or in three-dimensions, or a cut volume with a general shape.

34. (canceled)

35. (canceled)

36. The system of claim 20, wherein the CAS device is at least one of an active surgical robot, a semi-active surgical robot, or a haptic surgical robot.

37. (canceled)
